(19) United States
(12) Patent Application Publication
(10) Pub. No.: US 2024/0197173 A1
(43) Pub. Date: Jun. 20, 2024

(54) Ophthalmic Microscope System and corresponding System, Method and Computer Program

(71) Applicant: Leica Instruments (Singapore) Pte. Ltd., Singapore (SG)

(72) Inventors: Alvin KOK, Singapore (SG); Gao YANG, Singapore (SG); Zheng-Dong ZENG, Singapore (SG); Jiahao PAN, Singapore (SG)

(21) Appl. No.: 18/555,263

(22) PCT Filed: Apr. 7, 2022
(86) PCT No.: PCT/EP2022/059245

§ 371 Date: Oct. 13, 2023

(30) Foreign Application Priority Data
Apr. 13, 2021 (DE) 10 2021 109 118.7

(51) Int. Cl. A61B 3/13; A61B 3/00; A61B 90/00

(52) U.S. Cl. CPC A61B 3/13; A61B 3/0025; A61B 90/37; A61B 2090/3735

(57) ABSTRACT

Examples relate to an ophthalmic microscope system and to a corresponding system, method and computer program for an ophthalmic microscope system. The system comprises one or more processors and one or more storage devices. The system is configured to obtain intraoperative sensor data of an eye from at least one imaging device of the ophthalmic microscope system. The system is configured to process the intraoperative sensor data using a machine-learning model. The machine-learning model is trained to output information on one or more anatomical features of the eye based on the intraoperative sensor data. The system is configured to generate a display signal for a display device of the ophthalmic microscope system based on the information on the one or more anatomical features of the eye. The display signal comprises a visual guidance overlay for guiding a user of the ophthalmic microscope system with respect to the one or more anatomical features of the eye.

### TECHNICAL FIELD

Examples relate to an ophthalmic microscope system and to a corresponding system, method and computer program for an ophthalmic microscope system, more specifically, but not exclusively, to a concept for generating a visual guidance overlay for guiding a user of the ophthalmic microscope system.

### BACKGROUND

The visualization of tissue structures is a major focus in surgical microscopy. However, there may be inherent challenges with the visualization of such tissue structure through oculars of the respective surgical microscope systems. For example, in cataract surgery being performed with the help of an ophthalmic microscope systems, ophthalmologists often rely on the so-called red reflex, which provides ideal contrast to visualize the capsule, lens and anterior chamber structure. The red reflex may provide the necessary contrast between the lens and the posterior capsule of the eye, which provides information on the depth in which the surgeon is working within the eye. In dense cataracts however, the penetration of the red reflex light can be blocked by the opacity of the cataract lens, limiting the intensity of the red reflex observed.

The posterior face of the lens capsule provides a barrier between anterior and posterior segments during cataract surgery. Accidental tearing of the posterior capsule during cataract surgery complicates lens removal, hampers the insertion of implant lenses and results in a higher rate of postoperative issues. However, surgeons sometimes struggle with gauging the depth and strength of the membrane-like collagen structure, especially with poor red reflex illumination.

Retinal surgeries in the posterior of the eye often involve peeling procedures, for example, the peeling of the epiretinal membrane (ERM) or internal limiting membrane (ILM) to treat various vitreoretinal disorders including macular holes, macular puckers, epiretinal membranes, diabetic macular edema, retinal detachment. As both retinal membranes are semi-translucent and microns in thickness, surgeons often use toxic dyes such as Trypan blue or Indocyanine green (ICG) to stain and visualize the membranes.

The removal of vitreous (fluid) is another vital workflow step to prevent the reoccurrence of retinal detachment in the patient. Surgeons sometimes use steroids to stain the transparent vitreous white, to ensure the complete removal of vitreous pockets in the eye. However, these steroids may be similarly toxic to the patient and surgeons often limit or try avoiding entirely the use of these in surgeries where possible.

Viewing in the posterior may be further compounded by poor or low lighting from the endo-illumination system and semi-translucent tissue features, resulting in challenges in differentiating ocular features.

Image recognition, ratification intelligence and machine-learning have been used for the analysis of images in the medical fields, and in particular also for images that show ocular structures, to identify said ocular structures. However, such systems are merely used on static data, e.g., to annotate static images of ocular structures.

Some intraocular lens (IOL) guidance systems have some image recognition capabilities limited to identifying pupil sizes and scleral blood vessels tortuosity and thickness to enable the correct positioning of intraocular lens during cataract surgery. However, such systems are limited to pre-operative planning and aligning toric IOLs, and generally do not provide features to identify and track ocular features of interest intraoperatively.

There may be a desire for an improved concept for an ophthalmic microscope system.

### SUMMARY

This desire is addressed by the subject-matter of the independent claims.

Various embodiments of the present disclosure are based on the finding that machine-learning-based analysis of intraoperative sensor data of at least one imaging sensor of an ophthalmic microscope system can be employed to guide the surgeon during the surgical procedure.

Anatomical features are identified and tracked in the intraoperative sensor data using machine-learning, e.g., to classify and locate the anatomical features, and/or to detect anomalies with respect to the anatomical features. To guide the surgeon during surgery, a visual overlay is generated that is used to highlight or annotate anatomical features of interest in a visual representation of the intraoperative sensor data. For example, some anatomical structures may be highlighted, e.g., to highlight the edge of the afore-mentioned posterior capsule, or to annotate anomalies, such as holes in the retina or an incorrect orientation of a cornea graft. In effect, the proposed concept provides a method for identifying ocular features of interest, such as the posterior capsule, retinal membrane or macula holes, using digital augmentation to highlight and track the feature or features in a surgical display.

Various examples of the present disclosure relate to a system for an ophthalmic microscope system. The system comprises one or more processors and one or more storage devices. The system is configured to obtain intraoperative sensor data of an eye from at least one imaging device of the ophthalmic microscope system. The system is configured to process the intraoperative sensor data using a machine-learning model. The machine-learning model is trained to output information on one or more anatomical features of the eye based on the intraoperative sensor data. The system is configured to generate a display signal for a display device of the ophthalmic microscope system based on the information on the one or more anatomical features of the eye. The display signal comprises a visual guidance overlay for guiding a user of the ophthalmic microscope system with respect to the one or more anatomical features of the eye. For example, the machine-learning model may be used to track the anatomical features of the eye (i.e., the ocular features) in real-time, while the visual guidance overlay may be used to annotate or highlight at least some of the anatomical features to aid the surgeon during surgery.

In general, the surgeon may be guided by annotating the one or more anatomical features of the eye, e.g., by providing a textual annotation, by highlighting the anatomical features, by highlighting/tracing the edges of the one or more anatomical features, or by highlighting anomalies with respect to the one or more anatomical features. Accordingly, the visual guidance overlay may comprise an annotation of the one or more anatomical features of the eye, suitable for guiding a user of the ophthalmic microscope system during a surgical procedure.

During ophthalmic surgery, the condition of the eye continuously changes due to the operations being performed by the surgeon. Therefore, the intraoperative sensor data may be updated and processed continuously, and the visual guidance overlay may be updated accordingly. In other words, the system may be configured to obtain the intraoperative sensor data as a continuously updated stream of intraoperative sensor data. The system may be configured to update the visual guidance overlay based on the continuously updated stream of intraoperative sensor data.

The visual guidance overlay is used for guiding a user of the ophthalmic microscope system with respect to the one or more anatomical features of the eye. Accordingly, the visual guidance overlay may be overlaid over the intraoperative sensor data, or rather a visual representation thereof. In other words, the system may be configured to overlay the visual guidance overlay over a visual representation of the intraoperative sensor data within the display signal.

As mentioned above, the one or more anatomical features may be annotated within the visual guidance overlay. The system may be configured to generate the visual guidance overlay with one or more of a plurality of visual indicators. For example, the plurality of visual indicators comprising one or more of a textual annotation of at least a subset of the one or more anatomical features, an overlay for highlighting one or more surfaces of the one or more anatomical features, an overlay for highlighting one or more edges of the one or more anatomical features, one or more directional indicators, and one or more indicators related to one or more anomalies regarding the one or more anatomical features. For example, a textual annotation may be used to label the one or more anatomical features, to describe an anomaly regarding the one or more anatomical features, or to describe a subsequent task during the surgical procedure. For example, an overlay for highlighting the surface or the edges of anatomical features may help the surgeon distinguishing the anatomical features in the visual representation of the intraoperative sensor data. Directional operators may guide the surgeon towards the location of further operations to be performed. Similarly, indicators related to an anomaly may be used to warn the surgeon of the anomaly, and to guide the surgeon towards the location of further operations to be performed.

However, in different surgical procedures, or at different stages of a surgical procedure, different anatomical features may be of interest to the surgeon. Therefore, only a subset of the anatomical features might be considered for the visual guidance overlay. In other words, the system may be configured to generate the visual guidance overlay based on a selection of a subset of the plurality of visual indicators. For example, the selection may be based on an input of a user of the ophthalmic microscope system. In other words, the user, e.g., the surgeon, may select the anatomical features or categories of anatomical features for the visual guidance overlay. Additionally or alternatively, the system may be configured to determine the selection based on a progress of an ophthalmic surgical procedure being performed with the help of the ophthalmic microscope system. In other words, an automated system may be used to track the progress of the surgical procedure, and to adjust the anatomical feature or features being considered based on the progress of the surgical procedure, which may reduce an overhead for the surgeon so they can concentrate on the surgical procedure.

A major tool during ophthalmic procedure is Optical Coherence Tomography (OCT), which is used to obtain a depth profile of the layers of the eye at one or more scanning lines. For example, the intraoperative sensor data may comprise intraoperative optical coherence tomography sensor data. The machine-learning model may be trained to output information on one or more layers of the eye based on the intraoperative optical coherence tomography sensor data. The system may be configured to generate the visual guidance overlay with a visual indicator highlighting or annotating at least a subset of the one or more layers of the eye. For example, the layers of the eye may be hard to distinguish visually via intraoperative imaging sensor data, so the OCT is used to guide the surgeon, depth-wise, during surgery. By annotating/highlighting features shown in the intraoperative OCT sensor data, this navigation may be facilitated, and anomalies may be brought to the attention of the surgeon.

In some examples, the machine-learning model is trained to output information on a classification of the one or more anatomical features within the intraoperative sensor data. The system may be configured to generate the visual guidance overlay with a visual indicator related to the classification of the one or more anatomical features. For example, this may be used to aid the surgeon in distinguishing anatomical features in the visual representation of the intraoperative sensor data.

As mentioned above, the proposed concept may be used to detect and highlight anomalies. Accordingly, the machine-learning model may be trained to output information on one or more anomalies regarding the one or more anatomical features of the eye. The system may be configured to generate the visual guidance overlay with a visual indicator related to the one or more anomalies. For example, a warning message may be shown when an anomaly is detected. In other words, the system may be configured to include an alert on the one or more anomalies within the display signal, or to output the alert via an output device of the ophthalmic microscope system. Additionally or alternatively, a location of the anomaly may be highlighted in the visual representation of the intraoperative sensor data, or the anomality may be added to a list of tasks to perform by the surgeon.

In some examples, the system may comprise multiple imaging devices, such as an imaging sensor of a microscope of the ophthalmic microscope system and the aforementioned OCT system. In many cases, the intraoperative sensor data of one of the imaging devices may be more suitable for detecting a given anatomical feature. For example, intraoperative OCT sensor data is particularly suitable for detecting and distinguishing layers of the eye. If an anomaly is detected with respect to one of the layers of the eye in the intraoperative OCT sensor data, for example, this anomaly might not only be highlighted in a visual representation of the intraoperative OCT sensor data, but also (or exclusively) at a corresponding position of a visual representation of the intraoperative sensor data of the imaging sensor of the microscope. In more general terms, the intraoperative sensor data may comprise first intraoperative sensor data from a first imaging device and second intraoperative sensor data from a second imaging device. The system may be configured to generate the display signal with a first visual representation of the first intraoperative sensor data and with a second visual representation of the second intraoperative sensor data. The system may be configured to overlay a visual indicator of an anomaly detected by the machine-learning model based on the first intraoperative sensor data over a corresponding position of the second visual representation of the second intraoperative sensor data within the display signal. For example, corresponding visual indicators (e.g., having the same shape, color and/or line style) may be overlaid over both visual representations, so the surgeon can recognize the correspondence between the visual indicators.

Another aspect that may be used to guide the surgeon and facilitate surgery is the tracking of surgical instruments relative to the one or more anatomical features. For example, a distance between a surgical instrument and an anatomical feature may be tracked so the surgeon is able to operate in close proximity to the edge of the anatomical feature, without causing unwanted incisions. Accordingly, the system may be configured to detect a presence of one or more surgical instruments in the intraoperative sensor data. The system may be configured to determine a distance between the detected one or more surgical instruments and the one or more anatomical features. The system may be configured to generate the visual guidance overlay with a visual indicator representing the distance between the detected one or more surgical instruments and the one or more anatomical features.

As outlined above, different types of intraoperative sensor data may be processed by the proposed concept. For example, the intraoperative sensor data may comprise one or more of intraoperative optical coherence tomography sensor data of an intraoperative optical coherence tomography device of the ophthalmic microscope system, intraoperative imaging sensor data of an imaging sensor of a microscope of the ophthalmic microscope system, and intraoperative endoscope sensor data of an endoscope of the ophthalmic microscope system. For example, different types of intraoperative sensor data are particularly suitable for detecting different types of anatomical features.

The generated display signal may be output via the display device of the ophthalmic microscope system. For example, the system may be configured to provide the display signal to the display device of the ophthalmic microscope system. For example, the display device may be one of a head-up display, a head-mounted display, a display mounted to a microscope of the ophthalmic microscope system, and ocular displays of the microscope of the ophthalmic microscope system.

Various aspects of the present disclosure relate to a corresponding ophthalmic microscope system comprising the at least one imaging device, the display device, and the system presented above. For example, the at least one imaging device may comprise at least one of an intraoperative optical coherence tomography device, an imaging sensor of a microscope, and an endoscope. For example, intraoperative sensor data of different imaging devices may be particularly suitable for detecting different types of anatomical features.

Various aspects of the present disclosure relate to a corresponding method for an ophthalmic microscope system. The method comprises obtaining intraoperative sensor data of an eye from at least one imaging device of the ophthalmic microscope system. The method comprises processing the intraoperative sensor data using a machine-learning model. The machine-learning model is trained to output information on one or more anatomical features of the eye based on the intraoperative sensor data. The method comprises generating a display signal based on the information on the one or more anatomical features of the eye. The display signal comprises a visual guidance overlay for guiding a user of the ophthalmic microscope system with respect to the one or more anatomical features of the eye.

Various aspects of the present disclosure relate to a corresponding computer program with a program code for performing the above method when the computer program is executed on a processor.

### SHORT DESCRIPTION OF THE FIGURES

Some examples of apparatuses and/or methods will be described in the following by way of example only, and with reference to the accompanying figures, in which:

FIG. 1a shows a block diagram of an example of a system for an ophthalmic microscope system;

FIG. 1b shows a schematic diagram of an example of a system for an ophthalmic microscope system in the context of components of the ophthalmic microscope system;

FIG. 1c shows a schematic diagram of an example of an ophthalmic microscope system;

FIGS. 2a and 2b show schematic drawings of examples of an annotation of anatomical features overlaid over a visual representation of camera sensor data;

FIG. 3a shows a schematic drawing of an example of a textual annotation of anatomical features overlaid over a visual representation of OCT sensor data;

FIG. 3b shows a schematic drawing of an example of a graphical annotation of anatomical features overlaid over a visual representation of OCT sensor data;

FIG. 3c shows a schematic drawing of an example of a visual representation of camera sensor data and a visual representation of OCT sensor data being shown side by side;

FIG. 4a shows a schematic drawing of an example of a textual annotation and graphical annotation of layers of an eye;

FIG. 4b shows a schematic drawing of an example of a graphical annotation of layers of an eye, overlaid over a visual representation of OCT sensor data;

FIGS. 5a and 5b show schematic drawings of an example of a graphical annotation highlighting a posterior capsule rapture;

FIG. 6a shows a schematic drawing of an example of a graphical annotation highlighting holes in a retina, overlaid over a visual representation of camera sensor data and a visual representation of OCT sensor data;

FIG. 6b shows a schematic drawing of an example of a graphical annotation highlighting anomalies in a cornea graft, overlaid over a visual representation of camera sensor data and a visual representation of OCT sensor data;

FIG. 6c shows a schematic drawing of an example of a graphical annotation highlighting an open incision wound, overlaid over a visual representation of camera sensor data and a visual representation of OCT sensor data;

FIG. 7 shows a schematic drawing of an example of a graphical annotation highlighting a relative distance between instrument tips and an anatomical feature;

FIG. 8 shows a flow chart of an example of a method for an ophthalmic microscope system; and

FIG. 9 shows a schematic diagram of a system comprising a microscope and a computer system.

### DETAILED DESCRIPTION

Various examples will now be described more fully with reference to the accompanying drawings in which some examples are illustrated. In the figures, the thicknesses of lines, layers and/or regions may be exaggerated for clarity.

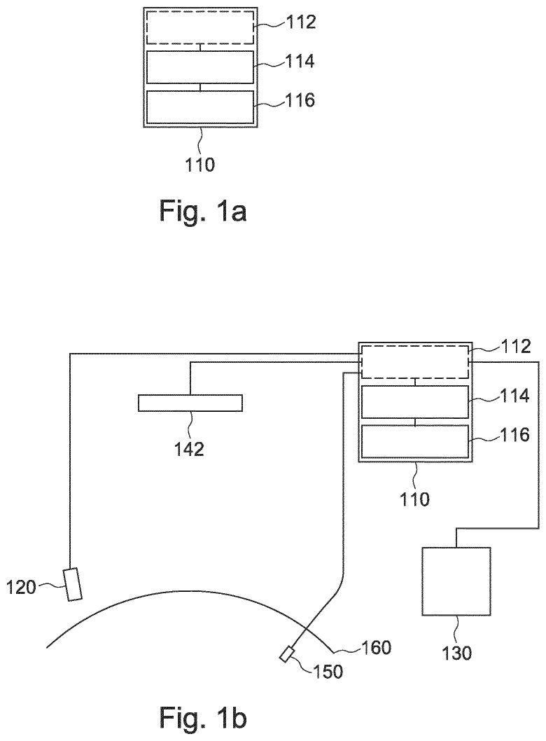

FIG. 1a shows a block diagram of an example of a system 110 for an ophthalmic microscope system (shown in FIG. 1c). Optionally, the system comprises an interface 112. The system 110 comprises one or more processors 114 and one or more storage devices 116. Optionally, the system comprises an interface 112. The one or more processors are coupled to the one or more storage devices and to the optional interface. In general, the functionality of the system is provided by the one or more processors, e.g., in conjunction with the optional interface (for exchanging information) and/or the one or more storage devices (for storing data).

The system is configured to obtain intraoperative sensor data of an eye from at least one imaging device 120; 142; 150 (as shown in FIGS. 1b and/or 1c) of the ophthalmic microscope system, e.g., via the interface 112. The system is configured to process the intraoperative sensor data using a machine-learning model. The machine-learning model is trained to output information on one or more anatomical features of the eye based on the intraoperative sensor data. The system is configured to generate a display signal for a display device 130; 130a; 130b; 130c (as shown in FIGS. 1b and/or 1c) of the ophthalmic microscope system based on the information on the one or more anatomical features of the eye. The display signal comprises a visual guidance overlay for guiding a user of the ophthalmic microscope system with respect to the one or more anatomical features of the eye.

In FIG. 1a, the system 110 is shown in isolation. However, the system 110 may be coupled to one or more optional components of the ophthalmic microscope system, as shown in FIG. 1b. FIG. 1b shows a schematic diagram of an example of the system for the ophthalmic microscope system in the context of components of the ophthalmic microscope system. For example, as shown in FIG. 1b, the system 110 may be coupled with the at least one imaging device of the ophthalmic microscope system, such as an intraoperative optical coherence tomography (intraoperative OCT or iOCT) device 120, an imaging sensor 142 of a microscope, and an endoscope 150 of the ophthalmic microscope system. For example, the imaging sensor 142 may be integrated in the microscope (140, as shown in FIG. 1c), while the OCT device 120 and the endoscope are used directly at, or even in, the eye 160.

FIG. 1c shows a schematic diagram of an example of the ophthalmic microscope system 100. The ophthalmic microscope system comprises at least one imaging device, such as the OCT device 120, the imaging sensor 142 of the microscope 140, or the endoscope 150, a display device 130a; 130b; 130c, and the system 110. In FIG. 1c, three potential display devices are shown—a head-up display 130a, ocular displays 130b of the microscope 140, and a display 130c that is mounted to the microscope (or rather a holding structure of the microscope). As the name indicates, the ophthalmic microscope system further comprises the microscope 140.

In general, a microscope, such as the microscope 140 shown in FIG. 1c, is an optical instrument that is suitable for examining objects that are potentially too small to be examined by the human eye (alone). For example, a microscope may provide an optical magnification of a sample. In modern microscopes, the optical magnification is often provided for a camera or an imaging sensor, such as the imaging sensor 142 of the microscope 140. In other words, the microscope 140 may be a digital microscope or a combined optical-digital microscope. Alternatively, a purely optical approach may be taken. The microscope 140 may further comprise one or more optical magnification components that are used to magnify a view on the sample, such as an objective (i.e., lens). In the context of this application, the term “ophthalmic microscope system” is used, in order to cover the portions of the system that are not part of the actual microscope (which comprises the optical components and is thus also denoted “optics carrier”), but which are used in conjunction with the microscope, such as the system 110, the OCT device 120, the display 130a-c or the endoscope 150.

The microscope system shown in FIG. 1c is an ophthalmic microscope system, which is a surgical microscope system for use during ophthalmic surgery, i.e., eye surgery. The ophthalmic microscope system 100 shown in FIG. 1c comprises a number of optional components, such as a base unit 105 (comprising the system 110) with a (rolling) stand, and a (robotic or manual) arm 170 which holds the microscope 140 in place, and which is coupled to the base unit 105 and to the microscope 140. As the present disclosure relates to an ophthalmic (surgical) microscope and ophthalmic microscope system for use in eye surgery, a sample being viewed through the microscope, is the eye 160, or at least the portion of the eye, of the patient.

Various examples of the present disclosure are used to generate a visual guidance overlay for guiding a user, e.g., the surgeon, of the ophthalmic microscope system with respect to the one or more anatomical features of the eye, e.g., by annotating the one or more anatomical features of the eye. This visual guidance overlay is, in turn, based on a machine-learning based analysis of the intraoperative sensor data of the at least one imaging sensor. Therefore, the system is configured to obtain the intraoperative sensor data of the eye from the at least one imaging device 120; 142; 150 of the ophthalmic microscope system. For example, the system may be configured to receive the intraoperative sensor data via the interface 112, i.e., the at least one imaging device may be configured to actively provide the intraoperative sensor data to the system. Alternatively, the system may be configured to read out the intraoperative sensor data from the at least one imaging device, or from a memory that is external to both the system and the at least one imaging sensor device.

In general, the proposed system is designed to be used during surgery—hence the usage of “intraoperative” sensor data. Accordingly, the intraoperative sensor data may be sensor data that is generated during a surgical procedure. For example, the intraoperative sensor data might not be sensor data that is collected before the start of the surgical procedure, e.g., not in preparation of the surgical procedure, but after a first incision has been made, for example. In various examples, the intraoperative sensor data is continuously updated—as the surgical procedure progresses, the intraoperative sensor data documents the progress of the surgical procedures. Therefore, the intraoperative sensor data may also be obtained, e.g., received or read out, continuously anew, so that the visual guidance overlay can be continuously re-generated based on up-to-date intraoperative sensor data. Consequently, the system may be configured to obtain the intraoperative sensor data as a continuously updated stream of intraoperative sensor data. Correspondingly, as is discussed in more detail below, the system may be configured to update the visual guidance overlay based on the continuously updated stream of intraoperative sensor data. In effect, the intraoperative sensor data, and the visual guidance overlay that is generated thereupon, may represent the eye being observed in (near) real-time (e.g., with at most 500 ms delay).

As mentioned above, the ophthalmic microscope system may comprise various imaging devices, such as the iOCT device 120, the imaging sensor 142 or the endoscope 150. The intraoperative sensor data may correspondingly comprise one or more of intraoperative optical coherence tomography sensor data of the intraoperative optical coherence tomography device 120 of the ophthalmic microscope system, intraoperative imaging sensor data of the imaging sensor 142 of the microscope 140 of the ophthalmic microscope system, and intraoperative endoscope sensor data of the endoscope 150 of the ophthalmic microscope system. In some cases, the intraoperative sensor data may comprise two or more sets of intraoperative sensor data, e.g., from two or more imaging sensors.

The intraoperative sensor data is processed using machine-learning, to determine information on one or more anatomical features of the eye based on the intraoperative sensor data. Accordingly, a method if provided for recognizing and digitally tracking anatomical (tissue) features using image recognition based on intraoperative sensor data, which may be provided by a camera (such as the imaging sensor of the microscope system), an intraoperative optical coherence tomography (iOCT) system, or by or other imaging accessories. Through referencing from a library of image and video datasets, which can be used to train the machine-learning model, software based on machine learning (ML) and particularly deep learning (DL) may be able to identify, localize and quantify anatomical or pathological features, such as differentiating the cornea, iris, anterior chamber angle, posterior capsule, etc. From intraoperative videos from the surgical camera, iOCT or other forms of imaging accessories, the software may be suitable for making inferences in real-time on which features are currently observed. The proposed concept thus provides a method for identifying ocular features of interest, such as the posterior capsule, retinal membrane or macula holes, and using digital augmentation to highlight and track the feature in the surgical display. In other words, various examples may perform digital visual tagging of tissue features.

Machine learning may refer to algorithms and statistical models that computer systems may use to perform a specific task without using explicit instructions, instead relying on models and inference. For example, in machine-learning, instead of a rule-based transformation of data, a transformation of data may be used, that is inferred from an analysis of historical and/or training data. For example, the content of images may be analyzed using a machine-learning model or using a machine-learning algorithm. In order for the machine-learning model to analyze the content of an image, the machine-learning model may be trained using training images as input and training content information as output. By training the machine-learning model with a large number of training images and/or training sequences (e.g., words or sentences) and associated training content information (e.g., labels or annotations), the machine-learning model “learns” to recognize the content of the images, so the content of images that are not included in the training data can be recognized using the machine-learning model. The same principle may be used for other kinds of sensor data as well: By training a machine-learning model using training sensor data and a desired output, the machine-learning model “learns” a transformation between the sensor data and the output, which can be used to provide an output based on non-training sensor data provided to the machine-learning model. The provided data (e.g., sensor data, meta data and/or image data) may be preprocessed to obtain a feature vector, which is used as input to the machine-learning model.

In the context of the present disclosure, the machine-learning model is trained to output the information on the one or more anatomical features of the eye based on the intraoperative sensor data. In other words, the intraoperative sensor data is provided at an input of the machine-learning model, and the information on the one or more anatomical features of the eye is provided at the output of the machine-learning model. The machine-learning model thus transforms the intraoperative sensor data into the information on the one or more anatomical features. In order to perform this transformation, the machine-learning model is trained on training data.

Machine-learning models may be trained using training input data. The examples specified above use a training method called “supervised learning”. In supervised learning, the machine-learning model is trained using a plurality of training samples, wherein each sample may comprise a plurality of input data values, and a plurality of desired output values, i.e., each training sample is associated with a desired output value. By specifying both training samples and desired output values, the machine-learning model “learns” which output value to provide based on an input sample that is similar to the samples provided during the training. Apart from supervised learning, semi-supervised learning may be used. In semi-supervised learning, some of the training samples lack a corresponding desired output value. Supervised learning may be based on a supervised learning algorithm (e.g., a classification algorithm, a regression algorithm or a similarity learning algorithm. Classification algorithms may be used when the outputs are restricted to a limited set of values (categorical variables), i.e., the input is classified to one of the limited set of values. Regression algorithms may be used when the outputs may have any numerical value (within a range). Similarity learning algorithms may be similar to both classification and regression algorithms but are based on learning from examples using a similarity function that measures how similar or related two objects are.

In the proposed concept, the machine-learning model is trained to process the intraoperative sensor data representing the one or more anatomical features in order to derive and output properties of the one or more anatomical features. For example, the one or more anatomical features may comprise one or more of at least one layer of the eye, at least one tissue structure of the eye, at least one graft, and at least one pathological feature. In general, there are (at least) four general categories of information on the one or more anatomical features that may be provided by the machine-learning model—an identity or classification of the one or more anatomical features, a location of the one or more anatomical features, a quantification of the one or more anatomical features, and anomalies among the one or more anatomical features.

To facilitate or enable the analysis of the one or more anatomical features by the machine-learning model, the machine-learning model may be trained to detect and segment the one or more anatomical features within the intraoperative sensor data. In other words, the machine-learning model may be trained to perform segmentation of the one or more anatomical features, in order to distinguish the individual anatomical features within the intraoperative sensor data. For example, supervised learning may be used to train the machine-learning model to perform segmentation, using samples of sensor data from the above-referenced sensors as training input data and manually segmented versions of the samples as desired output. The segmented anatomical features may subsequently be processed separately (or concurrently) by the machine-learning model. In some examples, the machine-learning model may comprise two or more sub-models—a first sub-model for performing the image segmentation, in order to isolate the one or more anatomical features, and one or more second sub-models for performing the further analysis of the segmented anatomical features.

For example, the machine-learning model may be trained to determine the identity of the one or more anatomical features. In machine-learning, this task is denoted a “classification” task. In other words, the machine-learning model may be trained to classify the one or more anatomical features, i.e., to output information on a classification of the one or more anatomical features within the intraoperative sensor data. As outlined above, supervised learning may be used to train the machine-learning model to perform the classification. For example, training samples representing anatomical features may be provided as training input, and information on a classification of the anatomical features represented in the training samples may be provided as desired output of the training of the machine-learning model. In effect, feature recognition is being performed on the intraoperative sensor data, and a method for identifying ocular features of interest, such as the posterior capsule, retinal membrane or macula holes is provided. As alluded earlier, the machine-learning model, or one of the second sub-models of the machine-learning model, may be trained to process each anatomical feature separately based on the segmentation of the one or more anatomical features.

In various examples, the location, and/or an extent, of the one or more anatomical features is output by the machine-learning model. For example, the location and/or extent of the one or more anatomical features may be output based on the segmentation of the one or more anatomical features. In other words, the machine-learning model may be trained to output one or more points representing the position and/or extent of the one or more anatomical features as a result of the segmentation. Similarly, the quantification of the one or more anatomical features may be performed based on the segmentation of the one or more anatomical features, e.g., during a post-processing task that may be used to determine the number and/or size of the respective segmented anatomical features.

As mentioned above, in some examples, the intraoperative sensor data comprises intraoperative optical coherence tomography sensor data. Optical coherence tomography is often used to scan the layers of the eye, providing a depth-analysis of the layers of the eye. These layers are represented by the intraoperative OCT sensor data and can be segmented and identified by the machine-learning model. Consequently, the machine-learning model may be trained to output information on one or more layers of the eye based on the intraoperative optical coherence tomography sensor data. To facilitate the processing of different types of intraoperative sensor data, the intraoperative sensor data may be input as image data into the machine-learning model. For example, the intraoperative OCT sensor data may be converted into image data, and provided as image data to the machine-learning model.

In this context, each layer of the eye may be considered a separate anatomical feature of the eye. Consequently, as outlined above, the machine-learning model may be trained to output information on a classification of the one or more layers corresponding to the one or more anatomical features of the eye. Similarly, the machine-learning model may be trained to output one or more points representing the position and/or extent of the one or more layers corresponding to the one or more layers corresponding to the one or more anatomical features of the eye.

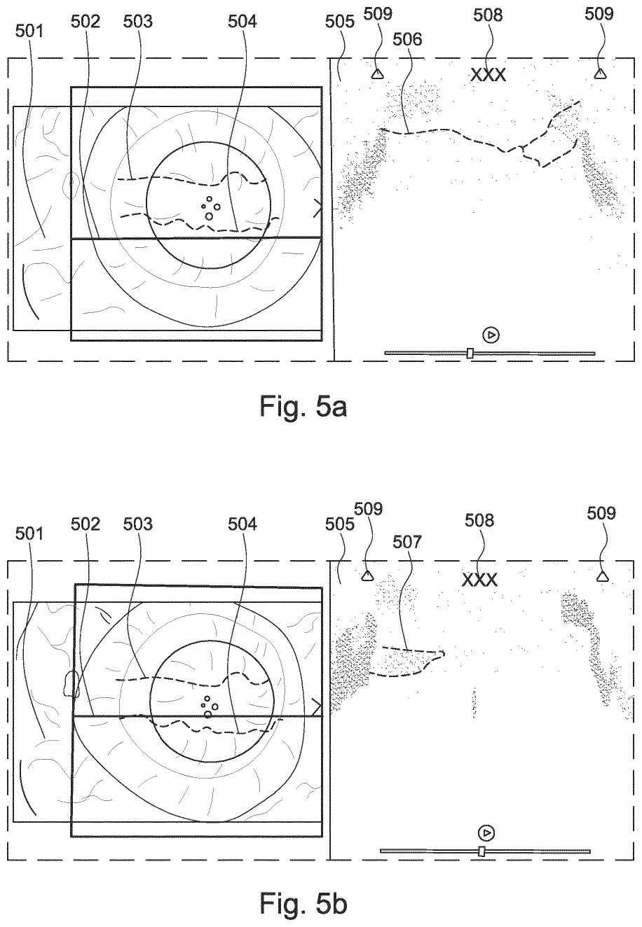

In some examples, the machine-learning model is further used for anomaly detection. In other words, the machine-learning model may be trained to output information on one or more anomalies regarding the one or more anatomical features of the eye. In this context, the anomaly detection may be used to identify at least one of two types of anomalies—anomalies regarding anatomical features to be treated as part of the surgical procedures, e.g., holes in the retina, as shown in connection with FIG. 6a, or incorrectly oriented cornea graft, as shown in connection with FIG. 6b, and anomalies regarding anatomical features that are an undesired or necessary byproduct of the surgical procedure, such as incision wounds, as shown in FIG. 6c. Again, supervised learning may be used to train the machine-learning model to output the information on the one or more anomalies regarding the one or more anatomical features of the eye. For example, samples of sensor data representing anomalies may be used as training samples, and the location of the anomalies and/or a classification of the anomalies may be used as desired output for the training of the machine-learning model. Consequently, the information on the one or more anomalies may comprise information on a location and/or a classification of the one or more anomalies. For example, one of the one or more second submodels may be trained to perform the anomaly detection separately on the segmented anatomical features.

In general, the machine-learning model may be used to classify, locate, quantify, or perform anomaly detection on, a variety of different types of anatomical features. However, in many cases, only a subset of the anatomical features may be of interest to the user, e.g., the surgeon. The user can either allow the software to automatically detect all tissue features or manually select features of interest to be identified. If only a subset of features is being of interest, the output of the machine-learning model may be filtered to (only) include anatomical features of interest, or another input may be provided to the machine-learning model that indicates the features of interest to the machine-learning model. This input may be considered in the training of the machine-learning model.

While the training of the machine-learning model is described in the context of the ophthalmic microscope system and corresponding system, method and computer program, the training of the machine-learning model may have concluded before the machine-learning model is loaded into the system and used to process the intraoperative sensor data. In other words, the machine-learning model may be a pre-trained machine-learning model that is trained by an entity that is external to the ophthalmic microscope system.

The output of the machine-learning model is used to generate the display signal with the visual guidance overlay. In other words, the system is configured to generate the display signal for the display device of the ophthalmic microscope system based on the information on the one or more anatomical features of the eye. In general, the display signal may be a signal for driving (e.g., controlling) the display. For example, the display signal may comprise video data and/or control instructions for driving the display. For example, the display signal may be provided via one of the one or more interfaces 112 of the system. Accordingly, the system 110 may comprise a video interface 112 that is suitable for providing the video signal to the display of the touch screen.

The display signal comprises a visual guidance overlay. As the name indicates, the visual guidance overlay may be overlaid over one or more other visual components of the display signal. For example, the display signal may further comprise a visual representation of the intraoperative sensor data. In other words, the system may be configured to overlay the visual guidance overlay over the visual representation of the intraoperative sensor data within the display signal. Moreover, a position of one or more elements, such as visual indicators, of the visual guidance overlay may be matched to corresponding portions of the intraoperative sensor data.

The system is configured to generate the visual guidance overlay as part of the display signal, with the visual guidance overlay being suitable for guiding the user of the ophthalmic microscope system with respect to the one or more anatomical features of the eye. This may be done by annotating the intraoperative sensor data with respect to the one or more anatomical features, with the visual guidance overlay comprising the annotation of the intraoperative sensor data. In other words, the visual guidance overlay may comprise an annotation of the one or more anatomical features of the eye, suitable for guiding a user of the ophthalmic microscope system during a surgical procedure. For example, digital augmentation may be used to highlight and track anatomical features in the surgical display.

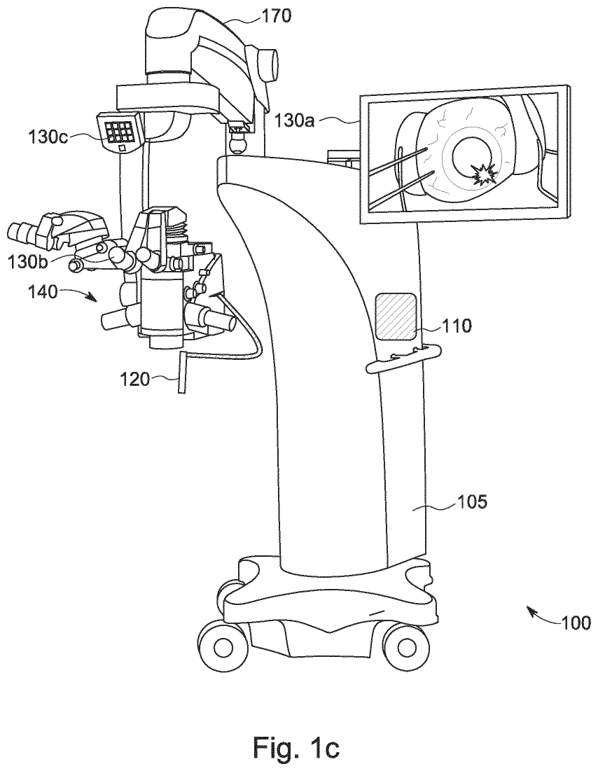

For example, the annotation may be performed by including one or more visual indicator in the visual guidance overlay. In other words, the system may be configured to generate the visual guidance overlay with one or more of a plurality of visual indicators. The one or more visual indicators may be used to annotate the one or more anatomical features. Therefore, the one or more visual indicators may be overlaid over the one or more anatomical features in the display signal, e.g., such that the position of the visual indicator in the visual guidance overlay matches the position of the corresponding anatomical feature shown in the representation of the intraoperative sensor data. Such an approach is applicable for various types of visual indicators. For example, the plurality of visual indicators may comprise one or more of an overlay for highlighting one or more surfaces of the one or more anatomical features, an overlay for highlighting one or more edges of the one or more anatomical features, one or more directional indicators, and one or more indicators related to one or more anomalies regarding the one or more anatomical features. These types of visual indicators may be overlaid directly over the corresponding anatomical feature shown in the visual representation of the intraoperative sensor data. In some examples, the plurality of visual indicators may comprise a textual annotation of at least a subset of the one or more anatomical features. As shown in FIGS. 2a, 2b, 3a and 4a, such textual annotations may be shown overlaid near the respective anatomical feature shown in the visual representation of the intraoperative sensor data, e.g., linked by a visual element, or may be shown as general information as part of a user interface that is part of the display signal.

As mentioned above, the intraoperative sensor data may be continuously updated during the surgical procedure. Correspondingly, the system may be configured to update the visual guidance overlay based on the continuously updated stream of intraoperative sensor data. For example, the system may be configured to periodically re-generate the visual guidance overlay based on the continuously updated stream of intraoperative sensor data. For example, the continuously updated stream of intraoperative sensor data may comprise a sequence of samples of intraoperative sensor data. The system may be configured to process at least a subset of the samples of the sequence of samples using the machine-learning model (e.g., every n-th sample, or according to a pre-defined frequency), and to re-generate the visual guidance display based on the latest output of the machine-learning model.

In the following, some examples are provided that illustrate the use of visual indicators to guide the surgeon during the surgical procedure.

As has been outlined with respect to the machine-learning model, the machine-learning model may be trained to output information on a classification of the one or more anatomical features within the intraoperative sensor data. This classification may be used to populate the visual guidance overlay, in order to provide a textual or graphical annotation of the respective anatomical features. In other words, the system may be configured to generate the visual guidance overlay with a visual indicator related to the classification of the one or more anatomical features. Examples for such a visual indicator related to the classification are given in FIGS. 2a to 4b.

FIGS. 2a and 2b show schematic drawings of examples of an annotation of anatomical features overlaid over a visual representation of camera sensor data. In FIGS. 2a and 2b, the software is used to identify, localize and quantify pathological features in the posterior and anterior. From intraoperative videos from the surgical camera, iOCT or other forms of imaging accessories, the software may make inferences in real-time on which features are currently observed. For example, in FIG. 2a, an annotation of the anatomical features that are visible in a camera image of the eye are shown. In FIG. 2a, a textual annotation may be overlaid over the camera image, as represented by the following numerals. For example, FIG. 2a shows an annotation of contraction furrows 201, pupil 202, collarette 203, crypts 204, ciliary zone 205, pupillary zone 206 and radial furrows 207. In FIG. 2b, anatomical features, and in particular anomalies with respect to anatomical features, such as hemorrhages and aneurysms are annotated as textual annotation. FIG. 2b shows an annotation of soft exudate 211, hemorrhages 212, micro-aneurysm 213, vasculature structure 214, hard exudate 215, optic disc 216, micro-hemorrhages 217 and macula 218 is shown.

A combination of inputs from both the camera, iOCT or other imaging accessories may be used to allow the software to interpret the anatomical details such as the size and depth of the structure. An example of the application of the proposed concept relates to the detection of the posterior capsule during cataract surgeries to help guide the surgeon's workflow to prevent the accidental tearing and rupture of the capsule when performing hydro-dissection, phacoemulsification and lens placement. This detection of the posterior capsule may be performed based on OCT sensor data, which can be used to distinguish the different layers of the eye. Accordingly, the system may be configured to generate the visual guidance overlay with a visual indicator highlighting or annotating at least a subset of the one or more layers of the eye. In effect, the information on the one or more layers of the eye, which is generated by the machine-learning model based on the intraoperative OCT sensor data, may be used to generate the visual guidance overlay with a visual indicator highlighting or annotating at least a subset of the one or more layers of the eye.

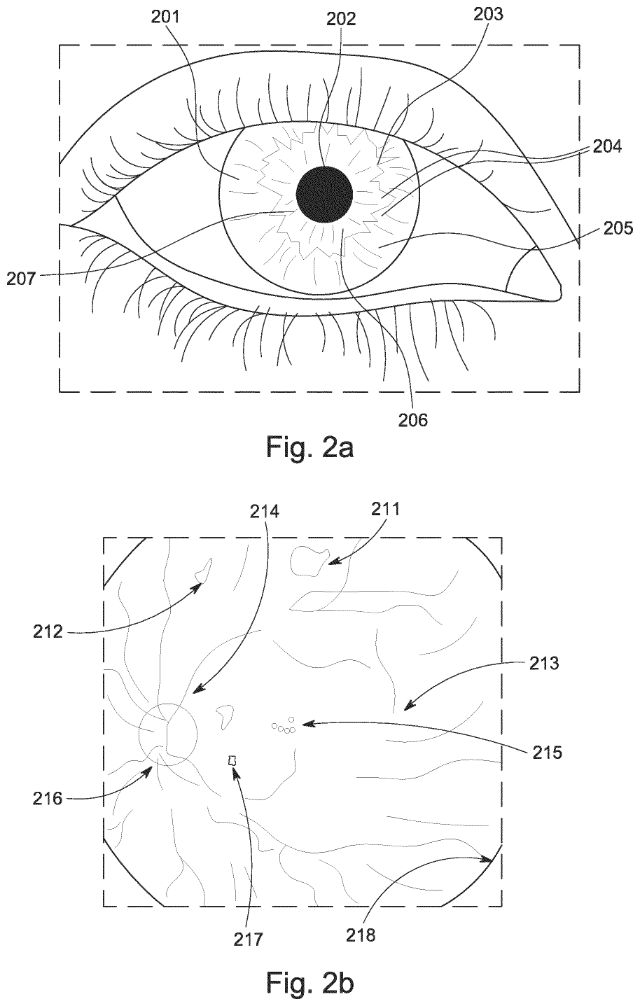

FIGS. 3a to 3c illustrate the detection of the posterior capsule during to prevent the accidental tearing and rupture of the capsule. In particular, FIG. 3a shows a schematic drawing of an example of a textual annotation of anatomical features overlaid over a visual representation of intraoperative OCT sensor data. In FIG. 3a, a textual annotation is used to annotate the anterior capsule 301, the IOL 302, and the posterior capsule 303.

In FIG. 3b, a different approach is chosen. FIG. 3b shows a schematic drawing of an example of a graphical annotation of anatomical features overlaid over a visual representation of OCT sensor data. In FIG. 3b, two visual indicators highlighting the edges of anatomical features are included the visual guidance overlay, a line 311 highlighting an edge of the IOL, and a line 312 highlighting an edge of the posterior capsule.

In FIG. 3c, an example of is given of a display signal comprising two visual representations 321; 323 of intraoperative sensor data. FIG. 3c shows a schematic drawing of an example of a visual representation of camera sensor data and a visual representation of OCT sensor data being shown side by side. On the left side, a visual representation 321 of intraoperative imaging sensor data of the imaging sensor of the microscope is shown (in the following denoted “camera view”), and on the right side, a visual representation 323 of intraoperative OCT sensor data is shown (in the following denoted “OCT view”). Over the camera view 321, a current scanning line 322 of the OCT is overlaid. As will be appreciated by those in the art, OCT is a scanning technique for taking three-dimensional scans of a target. However, the visual representation 323 only shows a two-dimensional visual representation of a cross-section of the three-dimensional scan. The OCT scanning line 322 indicates the position, of which the cross-section shown in the visual representation 323 of the intraoperative OCT sensor data is shown. The scanning line can be moved by the user, e.g., by moving the scanning line directly, or via a slider control that is shown underneath the visual representation 323 of the intraoperative OCT sensor data.

A variety of tissue structures can be identified intraoperatively and in real-time. To prevent information overload, in some examples, users may additionally select only to view specific features of interest relevant to their workflow step. In other words, the system may be configured to generate the visual guidance overlay based on a selection of a subset of the plurality of visual indicators. For example, a subset of the one or more anatomical features, and thus the subset of the plurality of visual indicators, may be selected by the user. Additionally or alternatively, the user may select a subset of types or causes of visual indicators to determine the plurality of visual indicators. Accordingly, the selection of the subset of the plurality of visual indicators may be based on an input of a user of the ophthalmic microscope system. This can be done either via the interactive graphic user interface, or via the microscope handles and footswitch.

The selected tissue features may then be augmented digitally in the surgical display, either by tracing over the edges, providing anatomical information, highlighting the structure, providing directional information, or other forms of information to guide the surgical workflow.

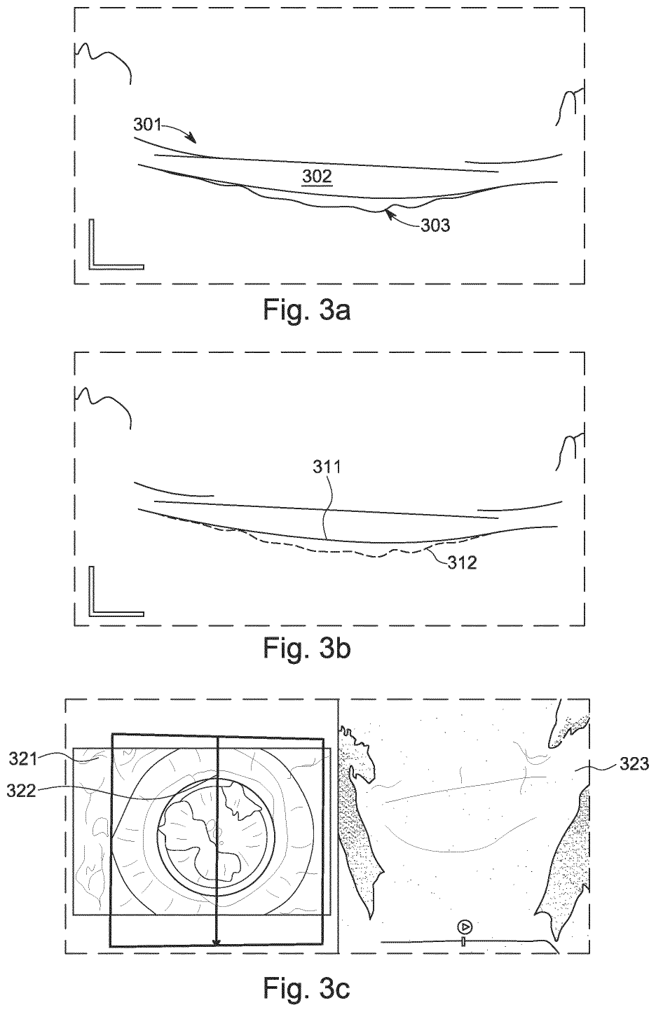

In FIGS. 4a to 4b, examples are given on how users can select to only view specific features of interest relevant to their workflow step. FIG. 4a shows a schematic drawing of an example of a textual annotation and graphical annotation of layers of an eye. FIG. 4a shows the layers Internal Limiting Membrane (ILM) 401, Retinal Nerve Fiber Layer (RNFL) 402, Ganglion Cell Layer (GCL) 403, Inner Plexiform Layer (IPL) 404, Inner Nuclear Layer (INL) 405, Outer Plexiform Layer (OPL) 406, Outer Nuclear Layer (ONL) 407, External Limiting Membrane (ELM) 408, Photoreceptor layers (PR) 409, Retinal Pigment Epithelium (RPE) 410, Bruch's Membrane (BM) 411, Choriocapillaris (CC) 412 and Choroidal Stroma (CS) 413. For example, the acronym of the respective layers may be shown on either side of the highlighted layers. For example, the user may select the layers of interest, e.g., by selecting or deselecting the layers in an illustration similar to the one shown in FIG. 4a.

Alternatively, the layers of interest, or more generally anatomical features of interest, may be selected automatically based on the progress of the surgical procedure. For example, the system may be configured to determine the selection of the subset of the plurality of visual indicators based on a progress of an ophthalmic surgical procedure being performed with the help of the ophthalmic microscope system. For example, the system may be configured to track the progress of the ophthalmic surgical procedure, e.g., based on a pre-operative plan, and to select a pre-defined set of visual indicators for the subset of the plurality of visual indicators.

A result of the selection, by the user, or by the system, is shown in FIG. 4b. FIG. 4b shows a schematic drawing of an example of a graphical annotation of layers of an eye, overlaid over a visual representation of OCT sensor data. In FIG. 4a, only a subset of the layers (Photoreceptor layer 424, Internal Limiting Membrane 425, Retinal Nerve Fiber 426 and Outer Nuclear Layer 427) are highlighted by a line trace. FIG. 4b further shows the camera view 421 and the OCT view 423 on the eye, and the position of the OCT scanning line 422.

Additionally, warnings may be provided on-screen to provide alerts to possible complications, e.g., due to abnormal tissue structures or potential errors in surgical techniques. Accordingly, as mentioned above, the machine-learning model may be trained to output information on one or more anomalies regarding the one or more anatomical features of the eye. The system may be configured to generate the visual guidance overlay with a visual indicator related to the one or more anomalies. In other words, the visual guidance overlay may comprise a visual indicator that warns or notifies the user of the one or more anomalies. For example, the visual indicator may comprise one or more of a warning message, a warning pictogram, and/or a visual indicator outlining a location of the anomaly. Accordingly, the system may be configured to include an alert on the one or more anomalies within the display signal. Alternatively or additionally, the system may be configured to output the alert via an output device of the ophthalmic microscope system, such as a warning light or a loudspeaker.

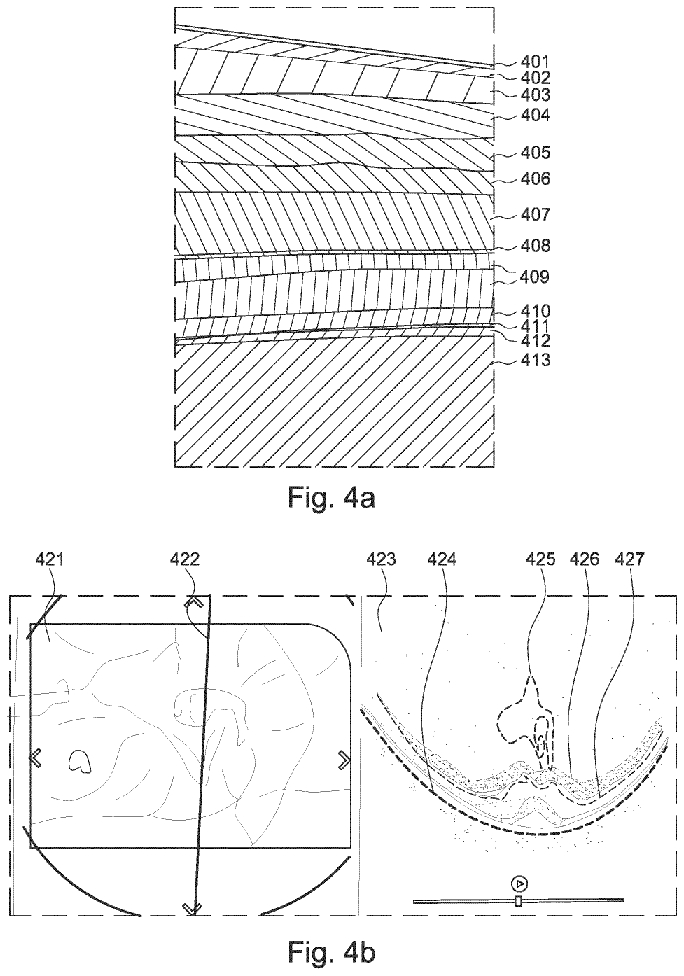

As shown in FIGS. 5a and 5b, warnings may be provided on-screen to provide alerts to possible complications due to abnormal tissue structures. A useful application of such warnings lies in a detection of an abnormal deformation in a shape or in a detection of micro-tears in the posterior capsule, showing an elevated risk of posterior capsule rupture when performing hydro-dissection, phacoemulsification and lens placement. FIGS. 5a and 5b show schematic drawings of an example of a graphical annotation highlighting a posterior capsule rapture. FIG. 5a shows two views on the eye, a camera view 501 and an OCT view 505, with the OCT scanning line 502 being overlaid over the camera view 501. In the camera view, two lines 503; 504 are shown overlaid, highlighting a first and a second edge of an abnormal tissue structure, in this case a rupture in the posterior capsule. The corresponding raptures at two different OCT scanning positions are highlighted by lines 506; 507 in the OCT view 505. Additionally, a warning message 508 “posterior capsule rapture” may be shown, optionally along with one or more warning pictograms 509.

Furthermore, the system may provide surgical confirmation and navigation guidance if a specific procedure has completed. For example, remaining holes in the retina can be detected through image recognition using iOCT and highlighted in the surgical display for easy tracking. Another example relates to a cornea transplant procedure, where the proposed system may detect if the graft is in the correct orientation and aligned accurately over the transplant site. FIGS. 6a to 6c show corresponding examples.

FIG. 6a illustrates an example of a surgical confirmation and navigation guidance that is based on whether a specific procedure has been completed. In this example, remaining holes in the retina can be detected through image recognition using iOCT and highlighted in the surgical display for easy tracking FIG. 6a shows a schematic drawing of an example of a graphical annotation highlighting holes in a retina, overlaid over a visual representation of camera sensor data and a visual representation of OCT sensor data. FIG. 6a shows, similar to FIGS. 4b to 5b, two views on the eye, a camera view 601 and an OCT view 607. The scanning line 602 of the OCT is overlaid over the camera view 601. In FIG. 6a, a first and a second hole in the retina are highlighted by circles 603; 606a. A corresponding circle 606b highlights the second hole in the OCT view 607b. In addition, directional markers 604; 605 are shown for the two holes. For example, the directional markers may be used to guide the surgeon towards a position of the holes.

As can be seen in FIG. 6a, in some cases, where two or more sets of intraoperative sensor data are obtained from two or more imaging devices, an anomaly may be detected in one set of intraoperative sensor data, and an indicator may be overlaid over the visual representation of the other set of intraoperative sensor data. For example, in FIG. 6a, the anomaly is detected in the intraoperative OCT sensor data and overlaid over the visual representation of the intraoperative OCT sensor data and the intraoperative imaging sensor data of the imaging sensor of the microscope. In other words, the intraoperative sensor data may comprise first intraoperative sensor data from a first imaging device (e.g., intraoperative OCT sensor data) and second intraoperative sensor data from a second imaging device (e.g., intraoperative imaging sensor data). The system may be configured to generate the display signal with a first visual representation of the first intraoperative sensor data and with a second visual representation of the second intraoperative sensor data. For example, the camera view 601 of FIG. 6a may show the second visual representation of the second intraoperative sensor data, and the OCT view 607 of FIG. 6a may show the first visual representation of the first intraoperative sensor data. The system may be configured to overlay a visual indicator 603; 604; 605; 606a of an anomaly detected by the machine-learning model based on the first intraoperative sensor data over a corresponding position of the second visual representation 601 of the second intraoperative sensor data within the display signal, e.g., in addition to a visual indicator 606b of the anomaly that is overlaid over the visual representation 607 of the first intraoperative sensor data. Another example of the concept is shown in FIG. 6b.

FIG. 6b illustrates another example of a surgical confirmation and navigation guidance that is based on whether a specific procedure has been completed. In this example, the system recognizes and prompts if cornea graft is not in the correct orientation or aligned accurately. FIG. 6b shows a schematic drawing of an example of a graphical annotation highlighting anomalies in a cornea graft, overlaid over a visual representation of camera sensor data and a visual representation of OCT sensor data. FIG. 6b shows a camera view 611 and a first and a second OCT view 616; 617. As two OCT devices are used, a first scanning line 612 of the first OCT and a second scanning line 614 of the second OCT are shown. In this example, triangles 613a; 615a are used to highlight a first and a second anomaly in cornea graft overlaid over the camera view, while triangles 613b; 615b highlight the same anomalies in the first and second OCT view 616; 617. For example, corresponding visual indicators (i.e., the triangles) that are overlaid over different visual representations of intraoperative sensor data may have the same shape, the same color and/or the same line pattern.

FIG. 6c illustrates another example of a surgical confirmation and navigation guidance that is based on whether a specific procedure has been completed. In this example, the system recognizes and prompts if an incision wound is open and needs further hydration. FIG. 6c shows a schematic drawing of an example of a graphical annotation highlighting an open incision wound, overlaid over a visual representation of camera sensor data and a visual representation of OCT sensor data. FIG. 6c shows a camera view 621, a first OCT view 625 and a second OCT view 627. FIG. 6c further shows corresponding first and second OCT scanning lines 622; 623. In FIG. 6c, an ellipse 624a highlighting an open incision wound is overlaid over the camera view, and a corresponding ellipse 624b highlighting the open incision wound is overlaid over the OCT view. In addition, a warning message 626 “hydration needed, wound not closed” is displayed.

In some examples, instrument detection can also be incorporated, e.g., to track the relative distance between the instrument tips and tissue structures. For example, the shape profile of instruments can be identified to track the relative distance between the instrument tips and tissue structures. This may allow the surgeons to perform precise, visually guided manipulation tasks while tracking the depth and distance of their instruments relative to the tissue feature of interest.

Accordingly, the system may be configured to detect a presence of one or more surgical instruments in the intraoperative sensor data. For example, the system may be configured to detect the presence of the one or more surgical instruments using an object-detection algorithm, such as a visual object matching algorithm or a machine-learning model being trained to detect the one or more instruments in the intraoperative sensor data (e.g., using a supervised-learning-based training of the machine-learning model). For example, the machine-learning model that is used for processing the intraoperative sensor data may be further trained to detect and locate the one or more instruments in the intraoperative sensor data.

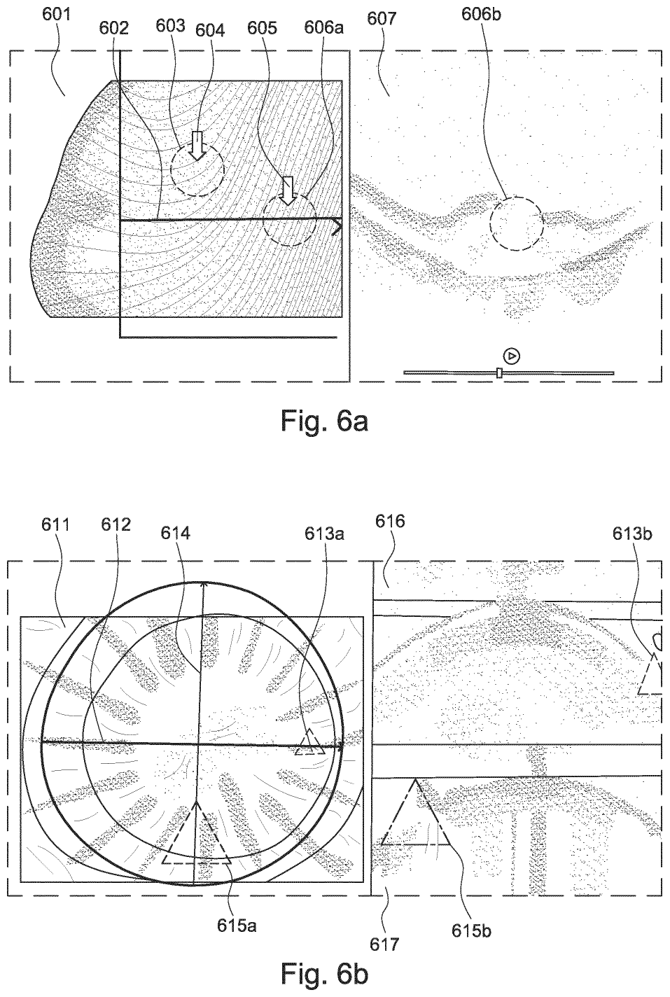

The system may be further configured to determine a distance between the detected one or more surgical instruments and the one or more anatomical features. For example, the intraoperative sensor data being used to detect the one or more surgical instruments, e.g., the intraoperative sensor data, as shown in FIG. 7, may have a known scale relative to the one or more surgical instruments, or the size of the one or more surgical instruments may be used to determine the scale of the intraoperative sensor data. The system may be configured to determine the distance between the detected one or more surgical instruments and the one or more anatomical features based on the scale of the intraoperative sensor data, and based on a distance (e.g., in pixels) between the detected one or more surgical instruments and the one or more anatomical features in a visual representation of the intraoperative sensor data. Alternatively, the distance may correspond to the distance (e.g., in pixels) between the detected one or more surgical instruments and the one or more anatomical features in a visual representation of the respective intraoperative sensor data. The system may be configured to generate the visual guidance overlay with a visual indicator representing the distance between the detected one or more surgical instruments and the one or more anatomical features. For example, the visual indicator representing the distance between the detected one or more surgical instruments may comprise a numerical representation of the distance, or the visual indicator may increase the contrast of the visual representation and/or highlight the edges of the one or more surgical instruments and/or of the one or more anatomical edges, to improve the visibility of the distance between the detected one or more surgical instruments may comprise a numerical representation of the distance. In some examples, if the distance is lower than a threshold, the visual indicator representing the distance may include a proximity warning.

FIG. 7 shows an example of how instrument detection can be incorporated to track the relative distance between the instrument tips and tissue structures. FIG. 7 shows a schematic drawing of an example of a graphical annotation highlighting a relative distance between instrument tips and an anatomical feature. FIG. 7a shows a camera view 701 and an OCT view 704. The instrument tips 705 are detected in the intraoperative OCT sensor data and highlighted in the visual representation of the visual sensor data. Circles 702-703 are used to highlight the instrument tips in the camera view and the OCT view. For example, FIG. 7a shows a circle 702a, highlighting a first instrument tip in the camera view, a corresponding circle 702b highlighting the first instrument tip in the OCT view, a circle 703a highlighting a second instrument tip in the camera view, and a corresponding circle 703b highlighting the second instrument tip in the OCT view. Similar to the examples shown in FIGS. 6a to 6c, corresponding visual indicators, such as circles, outlining the same instrument tip may have the same color, the same shape, and/or the same line pattern.

The display signal is generated for the display device of the ophthalmic microscope system. Correspondingly, the system may be configured to provide the display signal to the display device of the ophthalmic microscope system. For example, the surgical display may be in the form of a 3D heads-up surgical monitor, a head-mounted or microscope-mounted digital viewer or image injection into the microscope eyepieces. Accordingly, as illustrated in FIG. 1c, the display device may be one of a head-up display 130a (i.e., a display that is viewed by the surgeon while looking straight ahead, instead of down to the surgical site), a head-mounted display (not shown), such as virtual reality goggles or augmented- or mixed-reality glasses, a display 130c mounted to a microscope of the ophthalmic microscope system, and ocular displays 130b of the microscope of the ophthalmic microscope system.

The one or more interfaces 112 may correspond to one or more inputs and/or outputs for receiving and/or transmitting information, which may be in digital (bit) values according to a specified code, within a module, between modules or between modules of different entities. For example, the one or more interfaces 112 may comprise interface circuitry configured to receive and/or transmit information. In embodiments the one or more processors 114 may be implemented using one or more processing units, one or more processing devices, any means for processing, such as a processor, a computer or a programmable hardware component being operable with accordingly adapted software. In other words, the described function of the one or more processors 114 may as well be implemented in software, which is then executed on one or more programmable hardware components. Such hardware components may comprise a general-purpose processor, a Digital Signal Processor (DSP), a micro-controller, etc. In at least some embodiments, the one or more storage devices 116 may comprise at least one element of the group of a computer readable storage medium, such as a magnetic or optical storage medium, e.g., a hard disk drive, a flash memory, a Solid-State Disk (SSD), a Floppy-Disk, Random Access Memory (RAM), Programmable Read Only Memory (PROM), Erasable Programmable Read Only Memory (EPROM), an Electronically Erasable Programmable Read Only Memory (EEPROM), or a network storage.

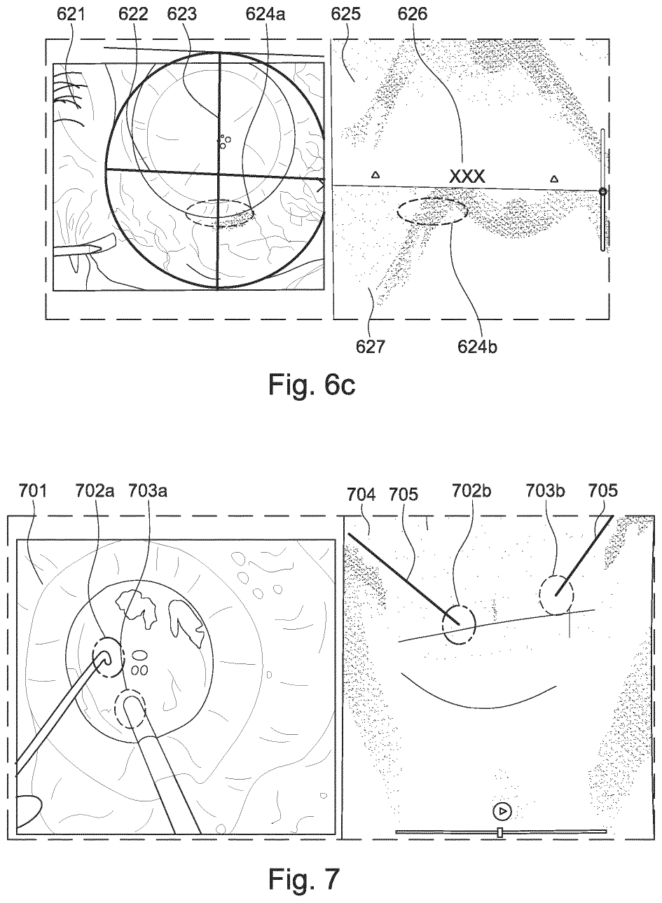

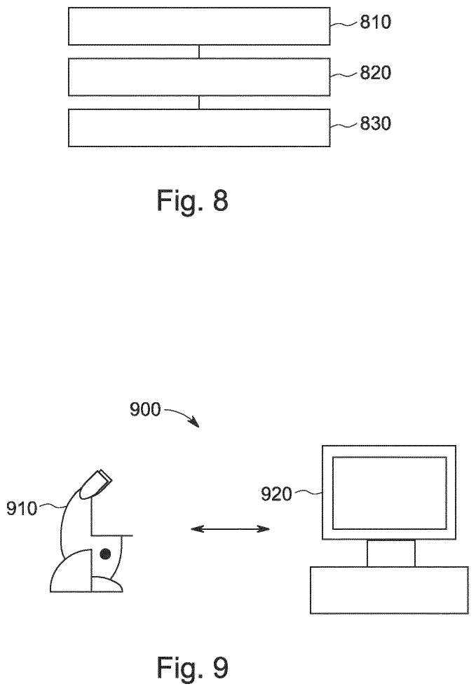

More details and aspects of the system and ophthalmic microscope system are mentioned in connection with the proposed concept or one or more examples described above or below (e.g., FIGS. 8 to 9). The system and ophthalmic microscope system may comprise one or more additional optional features corresponding to one or more aspects of the proposed concept or one or more examples described above or below.

FIG. 8 shows a flow chart of an example of a corresponding (computer-implemented) method for an ophthalmic microscope system, e.g., for the ophthalmic microscope system introduced in connection with FIGS. 1a to 7. For example, the method may be performed by the system 110 introduced in connection with FIGS. 1a to 7. The method comprises obtaining 810 intraoperative sensor data of an eye from at least one imaging device of the ophthalmic microscope system. The method comprises processing 820 the intraoperative sensor data using a machine-learning model. The machine-learning model is trained to output information on one or more anatomical features of the eye based on the intraoperative sensor data. The method comprises generating 830 a display signal based on the information on the one or more anatomical features of the eye. The display signal comprises a visual guidance overlay for guiding a user of the ophthalmic microscope system with respect to the one or more anatomical features of the eye.

Optionally, the method may comprise one or more further features, e.g., one or more features introduced with the system or surgical microscope system introduced in connection with FIGS. 1a to 7.

More details and aspects of the method are mentioned in connection with the proposed concept or one or more examples described above or below (e.g., FIGS. 1a to 7, 9). The method may comprise one or more additional optional features corresponding to one or more aspects of the proposed concept or one or more examples described above or below.

Some embodiments relate to a microscope comprising a system as described in connection with one or more of the FIGS. 1 to 8. Alternatively, a microscope may be part of or connected to a system as described in connection with one or more of the FIGS. 1 to 8. FIG. 9 shows a schematic illustration of a system 900 configured to perform a method described herein. The system 900 comprises a microscope 910 and a computer system 920. The microscope 910 is configured to take images and is connected to the computer system 920. The computer system 920 is configured to execute at least a part of a method described herein. The computer system 920 may be configured to execute a machine learning algorithm. The computer system 920 and microscope 910 may be separate entities but can also be integrated together in one common housing. The computer system 920 may be part of a central processing system of the microscope 910 and/or the computer system 920 may be part of a subcomponent of the microscope 910, such as a sensor, an actor, a camera or an illumination unit, etc. of the microscope 910.

The computer system 920 may be a local computer device (e.g., personal computer, laptop, tablet computer or mobile phone) with one or more processors and one or more storage devices or may be a distributed computer system (e.g., a cloud computing system with one or more processors and one or more storage devices distributed at various locations, for example, at a local client and/or one or more remote server farms and/or data centers). The computer system 920 may comprise any circuit or combination of circuits. In one embodiment, the computer system 920 may include one or more processors which can be of any type. As used herein, processor may mean any type of computational circuit, such as but not limited to a microprocessor, a microcontroller, a complex instruction set computing (CISC) microprocessor, a reduced instruction set computing (RISC) microprocessor, a very long instruction word (VLIW) microprocessor, a graphics processor, a digital signal processor (DSP), multiple core processor, a field programmable gate array (FPGA), for example, of a microscope or a microscope component (e.g., camera) or any other type of processor or processing circuit. Other types of circuits that may be included in the computer system 920 may be a custom circuit, an application-specific integrated circuit (ASIC), or the like, such as, for example, one or more circuits (such as a communication circuit) for use in wireless devices like mobile telephones, tablet computers, laptop computers, two-way radios, and similar electronic systems. The computer system 920 may include one or more storage devices, which may include one or more memory elements suitable to the particular application, such as a main memory in the form of random access memory (RAM), one or more hard drives, and/or one or more drives that handle removable media such as compact disks (CD), flash memory cards, digital video disk (DVD), and the like. The computer system 920 may also include a display device, one or more speakers, and a keyboard and/or controller, which can include a mouse, trackball, touch screen, voice-recognition device, or any other device that permits a system user to input information into and receive information from the computer system 920.

Some or all of the method steps may be executed by (or using) a hardware apparatus, like for example, a processor, a microprocessor, a programmable computer or an electronic circuit. In some embodiments, some one or more of the most important method steps may be executed by such an apparatus.

Depending on certain implementation requirements, embodiments of the invention can be implemented in hardware or in software. The implementation can be performed using a nontransitory storage medium such as a digital storage medium, for example a floppy disc, a hard disk drive (HDD), a solid state disk (SSD), a DVD, a Blu-Ray, a CD, a ROM, a PROM, and EPROM, an EEPROM or a FLASH memory, having electronically readable control signals stored thereon, which cooperate (or are capable of cooperating) with a programmable computer system such that the respective method is performed. Therefore, the digital storage medium may be computer readable.

Some embodiments according to the invention comprise a data carrier having electronically readable control signals, which are capable of cooperating with a programmable computer system, such that one of the methods described herein is performed.

Generally, embodiments of the present invention can be implemented as a computer program product with a program code, the program code being operative for performing one of the methods when the computer program product runs on a computer. The program code may, for example, be stored on a machine readable carrier.

Other embodiments comprise the computer program for performing one of the methods described herein, stored on a machine readable carrier.

In other words, an embodiment of the present invention is, therefore, a computer program having a program code for performing one of the methods described herein, when the computer program runs on a computer.

A further embodiment of the present invention is, therefore, a storage medium (or a data carrier, or a computer-readable medium) comprising, stored thereon, the computer program for performing one of the methods described herein when it is performed by a processor. The data carrier, the digital storage medium or the recorded medium are typically tangible and/or non-transitionary. A further embodiment of the present invention is an apparatus as described herein comprising a processor and the storage medium.

A further embodiment of the invention is, therefore, a data stream or a sequence of signals representing the computer program for performing one of the methods described herein. The data stream or the sequence of signals may, for example, be configured to be transferred via a data communication connection, for example, via the internet.

A further embodiment comprises a processing means, for example, a computer or a programmable logic device, configured to, or adapted to, perform one of the methods described herein.

A further embodiment comprises a computer having installed thereon the computer program for performing one of the methods described herein.

A further embodiment according to the invention comprises an apparatus or a system configured to transfer (for example, electronically or optically) a computer program for performing one of the methods described herein to a receiver. The receiver may, for example, be a computer, a mobile device, a memory device or the like. The apparatus or system may, for example, comprise a file server for transferring the computer program to the receiver.

In some embodiments, a programmable logic device (for example, a field programmable gate array) may be used to perform some or all of the functionalities of the methods described herein. In some embodiments, a field programmable gate array may cooperate with a microprocessor in order to perform one of the methods described herein. Generally, the methods are preferably performed by any hardware apparatus.

Embodiments may be based on using a machine-learning model or machine-learning algorithm. Two learning approaches, i.e., supervised learning and semi-supervised learning, have been discussed with respect to FIGS. 1a to 7.

Apart from supervised or semi-supervised learning, unsupervised learning may be used to train the machine-learning model. In unsupervised learning, (only) input data might be supplied and an unsupervised learning algorithm may be used to find structure in the input data (e.g., by grouping or clustering the input data, finding commonalities in the data). Clustering is the assignment of input data comprising a plurality of input values into subsets (clusters) so that input values within the same cluster are similar according to one or more (pre-defined) similarity criteria, while being dissimilar to input values that are included in other clusters.

Reinforcement learning is a third group of machine-learning algorithms. In other words, reinforcement learning may be used to train the machine-learning model. In reinforcement learning, one or more software actors (called “software agents”) are trained to take actions in an environment. Based on the taken actions, a reward is calculated. Reinforcement learning is based on training the one or more software agents to choose the actions such, that the cumulative reward is increased, leading to software agents that become better at the task they are given (as evidenced by increasing rewards).

Furthermore, some techniques may be applied to some of the machine-learning algorithms. For example, feature learning may be used. In other words, the machine-learning model may at least partially be trained using feature learning, and/or the machine-learning algorithm may comprise a feature learning component. Feature learning algorithms, which may be called representation learning algorithms, may preserve the information in their input but also transform it in a way that makes it useful, often as a pre-processing step before performing classification or predictions. Feature learning may be based on principal components analysis or cluster analysis, for example.

In some examples, anomaly detection (i.e., outlier detection) may be used, which is aimed at providing an identification of input values that raise suspicions by differing significantly from the majority of input or training data. In other words, the machine-learning model may at least partially be trained using anomaly detection, and/or the machine-learning algorithm may comprise an anomaly detection component.

In some examples, the machine-learning algorithm may use a decision tree as a predictive model. In other words, the machine-learning model may be based on a decision tree. In a decision tree, observations about an item (e.g., a set of input values) may be represented by the branches of the decision tree, and an output value corresponding to the item may be represented by the leaves of the decision tree. Decision trees may support both discrete values and continuous values as output values. If discrete values are used, the decision tree may be denoted a classification tree, if continuous values are used, the decision tree may be denoted a regression tree.

Association rules are a further technique that may be used in machine-learning algorithms. In other words, the machine-learning model may be based on one or more association rules. Association rules are created by identifying relationships between variables in large amounts of data. The machine-learning algorithm may identify and/or utilize one or more relational rules that represent the knowledge that is derived from the data. The rules may e.g., be used to store, manipulate or apply the knowledge.

Machine-learning algorithms are usually based on a machine-learning model. In other words, the term “machine-learning algorithm” may denote a set of instructions that may be used to create, train or use a machine-learning model. The term “machine-learning model” may denote a data structure and/or set of rules that represents the learned knowledge (e.g., based on the training performed by the machine-learning algorithm). In embodiments, the usage of a machine-learning algorithm may imply the usage of an underlying machine-learning model (or of a plurality of underlying machine-learning models). The usage of a machine-learning model may imply that the machine-learning model and/or the data structure/set of rules that is the machine-learning model is trained by a machine-learning algorithm.

For example, the machine-learning model may be an artificial neural network (ANN). ANNs are systems that are inspired by biological neural networks, such as can be found in a retina or a brain. ANNs comprise a plurality of interconnected nodes and a plurality of connections, so-called edges, between the nodes. There are usually three types of nodes, input nodes that receiving input values, hidden nodes that are (only) connected to other nodes, and output nodes that provide output values. Each node may represent an artificial neuron. Each edge may transmit information, from one node to another. The output of a node may be defined as a (non-linear) function of its inputs (e.g., of the sum of its inputs). The inputs of a node may be used in the function based on a “weight” of the edge or of the node that provides the input. The weight of nodes and/or of edges may be adjusted in the learning process. In other words, the training of an artificial neural network may comprise adjusting the weights of the nodes and/or edges of the artificial neural network, i.e., to achieve a desired output for a given input.

Alternatively, the machine-learning model may be a support vector machine, a random forest model or a gradient boosting model. Support vector machines (i.e., support vector networks) are supervised learning models with associated learning algorithms that may be used to analyze data (e.g., in classification or regression analysis). Support vector machines may be trained by providing an input with a plurality of training input values that belong to one of two categories. The support vector machine may be trained to assign a new input value to one of the two categories. Alternatively, the machine-learning model may be a Bayesian network, which is a probabilistic directed acyclic graphical model. A Bayesian network may represent a set of random variables and their conditional dependencies using a directed acyclic graph. Alternatively, the machine-learning model may be based on a genetic algorithm, which is a search algorithm and heuristic technique that mimics the process of natural selection.

As used herein the term “and/or” includes any and all combinations of one or more of the associated listed items and may be abbreviated as “/”.

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus.

### LIST OF REFERENCE SIGNS

100 Ophthalmic microscope system
105 Base unit
110 System
112 Interface
114 Processor
116 Storage device
120 OCT device
130 Display device
130a Head-up display
130b Ocular displays
130c Display arranged at microscope
140 Microscope
142 Optical imaging sensor
150 Endoscope
160 Eye
170 Arm
201-218 Textual annotation of anatomical features
301 Anterior Capsule
302 IOL
303 Posterior capsule
311 Line highlighting edge of IOL
312 Line highlighting posterior capsule
321 Camera view
322 Scanning line of OCT
323 OCT view
401-413 Layers of the eye
421 Camera view
422 Scanning line of OCT
423 OCT view
424-427 Lines highlighting layers of an eye
501 Camera view
502 Scanning line of OCT
503, 504 Line highlighting edges of abnormal tissue structure
505 OCT view
506, 507 Lines highlighting abnormal tissue structure in OCT
508 Warning message “posterior capsule rapture”
509 Warning pictogram
601 Camera view
602 Scanning line of OCT
603, 606a Circles highlighting holes in retina in camera view
604, 605 Directional markers
606b Circle highlighting hole in retina in OCT view
607 OCT view
611 Camera view
612, 614 Scanning lines of OCT
613a; 615a Triangles highlighting anomalies in cornea graft in camera view
613b; 615b Triangles highlighting anomalies in cornea graft in OCT view
616, 617 OCT views
621 Camera view
622, 623 OCT scanning lines
624a Ellipse highlighting open incision wound in camera view
624b Ellipse highlighting open incision wound in OCT view
625, 627 OCT views
626 Warning message “hydration needed, wound not closed”
701 Camera view
702a, 703a Circles highlighting instrument tips in camera view
702b, 703b Circles highlighting instrument tips in OCT view
704 OCT view
705 Instrument tips
810 Obtaining intraoperative sensor data
820 Processing the intraoperative sensor data using a machine-learning model
830 Generating a display signal with a visual guidance overlay
900 System
910 Microscope
920 Computer system

1. A system for an ophthalmic microscope system, the system comprising one or more processors and one or more storage devices, wherein the system is configured to:
obtain intraoperative sensor data of an eye from at least one imaging device of the ophthalmic microscope system;
process the intraoperative sensor data using a machine-learning model, the machine-learning model being trained to output information on one or more anatomical features of the eye based on the intraoperative sensor data; and
generate a display signal for a display device of the ophthalmic microscope system based on the information on the one or more anatomical features of the eye, the display signal comprising a visual guidance overlay for guiding a user of the ophthalmic microscope system with respect to the one or more anatomical features of the eye.

2. The system according to claim 1, wherein the visual guidance overlay comprises an annotation of the one or more anatomical features of the eye, suitable for guiding a user of the ophthalmic microscope system during a surgical procedure.

3. The system according to claim 1, wherein the system is configured to obtain the intraoperative sensor data as a continuously updated stream of intraoperative sensor data, and wherein the system is configured to update the visual guidance overlay based on the continuously updated stream of intraoperative sensor data.

4. The system according to claim 1, wherein the system is configured to overlay the visual guidance overlay over a visual representation of the intraoperative sensor data within the display signal.

5. The system according to claim 1, wherein the system is configured to generate the visual guidance overlay with one or more of a plurality of visual indicators, the plurality of visual indicators comprising one or more of a textual annotation of at least a subset of the one or more anatomical features, an overlay for highlighting one or more surfaces of the one or more anatomical features, an overlay for highlighting one or more edges of the one or more anatomical features, one or more directional indicators, and one or more indicators related to one or more anomalies regarding the one or more anatomical features.

6. The system according to claim 5, wherein the system is configured to generate the visual guidance overlay based on a selection of a subset of the plurality of visual indicators,
wherein the selection is based on an input of a user of the ophthalmic microscope system,
or wherein the system is configured to determine the selection based on a progress of an ophthalmic surgical procedure being performed with the help of the ophthalmic microscope system.

7. The system according to claim 1, wherein the intraoperative sensor data comprises intraoperative optical coherence tomography sensor data, wherein the machine-learning model is trained to output information on one or more layers of the eye based on the intraoperative optical coherence tomography sensor data, wherein the system is configured to generate the visual guidance overlay with a visual indicator highlighting or annotating at least a subset of the one or more layers of the eye.

8. The system according to claim 1, wherein the machine-learning model is trained to output information on a classification of the one or more anatomical features within the intraoperative sensor data, wherein the system is configured to generate the visual guidance overlay with a visual indicator related to the classification of the one or more anatomical features.

9. The system according to claim 1, wherein the machine-learning model is trained to output information on one or more anomalies regarding the one or more anatomical features of the eye, wherein the system is configured to generate the visual guidance overlay with a visual indicator related to the one or more anomalies.

10. The system according to claim 9, wherein the intraoperative sensor data comprises first intraoperative sensor data from a first imaging device and second intraoperative sensor data from a second imaging device, wherein the system is configured to generate the display signal with a first visual representation of the first intraoperative sensor data and with a second visual representation of the second intraoperative sensor data, wherein the system is configured to overlay a visual indicator of an anomaly detected by the machine-learning model based on the first intraoperative sensor data over a corresponding position of the second visual representation of the second intraoperative sensor data within the display signal.

11. The system according to claim 1, wherein the system is configured to detect a presence of one or more surgical instruments in the intraoperative sensor data, to determine a distance between the detected one or more surgical instruments and the one or more anatomical features, and to generate the visual guidance overlay with a visual indicator representing the distance between the detected one or more surgical instruments and the one or more anatomical features.

12. The system according to claim 1, wherein the intraoperative sensor data comprises one or more of intraoperative optical coherence tomography sensor data of an intraoperative optical coherence tomography device of the ophthalmic microscope system, intraoperative imaging sensor data of an imaging sensor of a microscope of the ophthalmic microscope system, and intraoperative endoscope sensor data of an endoscope of the ophthalmic microscope system.

13. An ophthalmic microscope system comprising at least one imaging device, a display device, and the system according to claim 1.

14. A method for an ophthalmic microscope system, the method comprising:
obtaining intraoperative sensor data of an eye from at least one imaging device of the ophthalmic microscope system;
processing the intraoperative sensor data using a machine-learning model, the machine-learning model being trained to output information on one or more anatomical features of the eye based on the intraoperative sensor data; and
generating a display signal based on the information on the one or more anatomical features of the eye, the display signal comprising a visual guidance overlay for guiding a user of the ophthalmic microscope system with respect to the one or more anatomical features of the eye.

15. (canceled)
